(19) United States
(12) Patent Application Publication
(10) Pub. No.: US 2025/0344969 A1
(43) Pub. Date: Nov. 13, 2025

(54) NONINVASIVE OPTICAL SYSTEM AND METHOD FOR MEASURING HEMOGLOBIN A1C AND OTHER BLOOD ANALYTES

(71) Applicant: Joseph David Allen, JR., Grand Forks, ND (US)

(72) Inventor: Joseph David Allen, JR., Grand Forks, ND (US)

(21) Appl. No.: 18/657,227

(22) Filed: May 7, 2024

(51) Int. Cl. A61B 5/1455; G16H 10/40; G16H 10/60; G16H 20/10; G16H 40/67

(52) U.S. Cl. CPC A61B 5/1455; G16H 10/40; G16H 10/60; G16H 20/10; G16H 40/67

(57) ABSTRACT

A noninvasive system and method estimate hemoglobin A1c, glucose, lipids, and other blood analytes without requiring a blood sample. The portable device uses diffuse reflectance spectroscopy and optical sensing across various wavelengths to collect data from the user's skin, interstitial fluid, saliva, sweat, tear fluid, or exhaled air. Machine learning algorithms analyze the optical data to estimate blood analyte levels, which are displayed on the device or synced with a software application. The system securely transmits data to the user's electronic health record for integration and remote monitoring. The invention encompasses FDA-approved, CE-marked, and non-FDA-approved devices, including key components, digital health features, and various medical applications.

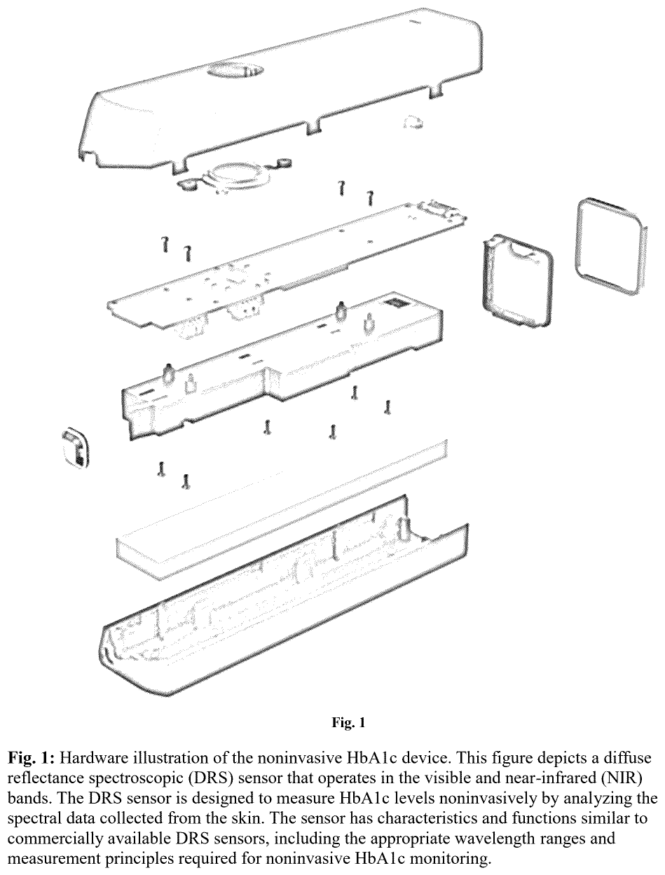

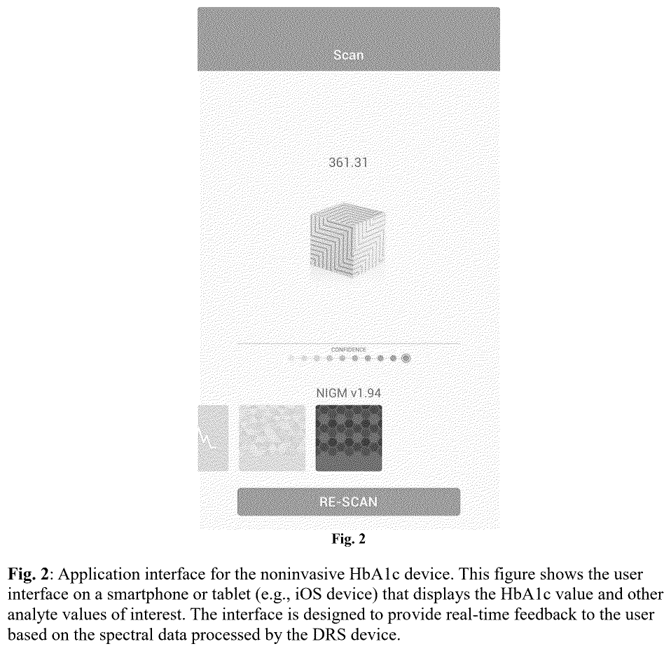

Abstract: A noninvasive system and method are provided for measuring hemoglobin A1c (HbA1c), glucose, lipids, and other blood analytes without requiring a blood sample. The system includes a portable device that uses diffuse reflectance spectroscopy (DRS) and other optical sensing methods across various electromagnetic wavelengths, including visible, ultraviolet, near-infrared, mid-infrared, and far-infrared regions, to collect data from a user's skin, interstitial fluid, saliva, sweat, tear fluid, or exhaled air. The optical data is analyzed by machine learning algorithms, including convolutional neural networks (CNNs), to estimate the user's blood analyte levels. The estimated values are displayed on the device's screen or synced with a software application compatible with smartphones, tablets, computers, and other devices. The system securely transmits the user's blood analyte data to their electronic health record, enabling integration with the healthcare provider's information systems and remote monitoring by physicians. The machine learning models are trained on datasets of optical scans paired with reference blood measurements to enable accurate noninvasive monitoring. The invention encompasses FDA-approved, CE-marked, and non-FDA-approved devices and includes the device itself, key components, digital health features, and use for various medical applications.

### DETAILED DESCRIPTION

### Field of the Invention

The present invention relates to systems and methods for noninvasive measurement of blood analytes, including hemoglobin A1c (HbA1c), glucose, lipids, and other components of a comprehensive metabolic panel (CMP). More specifically, the invention relates to a portable optical device that uses diffuse reflectance spectroscopy, machine learning algorithms, and a software application to estimate blood analyte levels without requiring a blood sample.

### BACKGROUND

Monitoring of HbA1c, glucose, lipids, and other blood analytes is important for managing diabetes, cardiovascular disease, and other health conditions. Conventional testing requires blood samples and laboratory analysis, which is inconvenient for frequent monitoring. There is a need for noninvasive monitoring methods to facilitate more regular testing by patients at home. Both FDA-approved and non-FDA-approved noninvasive devices are desirable to increase options for patients and providers.

### SUMMARY OF THE INVENTION

The present invention provides a noninvasive system and method for measuring HbA1c, glucose, lipids, and other blood analytes without requiring a blood sample. The system includes a portable device with optical sensors that collect data from the user's skin, interstitial fluid, saliva, sweat, tear fluid, or exhaled air using various electromagnetic wavelengths, including visible, ultraviolet, near-infrared, mid-infrared, and far-infrared regions. Machine learning algorithms analyze the optical data to estimate the user's blood analyte levels, which are displayed on the device's screen or synced with a software application compatible with smartphones, tablets, computers, and other devices.

In one aspect, the portable device includes light sources such as LEDs, lasers, or broadband emitters that illuminate the user's tissue and detectors that collect the diffusely reflected, transmitted, or emitted light at various wavelengths across the electromagnetic spectrum. The device also includes a microprocessor that runs the machine learning algorithms, a display, a wireless transceiver, and a rechargeable battery.

In another aspect, the machine learning algorithms may include convolutional neural networks (CNNs), support vector machines (SVMs), partial least squares regression (PLSR), random forests, or other models suitable for optical spectroscopy data. The models are trained on large datasets of optical scans from multiple anatomical sites and tissue types paired with reference capillary, venous, and arterial blood measurements. This allows the models to recognize the spectral features that correlate with blood analyte levels and output accurate estimations.

In yet another aspect, the invention includes a software application that pairs with the portable device to display blood analyte trends, provide guidance, and share data with healthcare providers. The application securely transmits the user's blood analyte data to their electronic health record, enabling integration with the healthcare provider's information systems and remote monitoring by physicians. The application may also allow manual entry of additional health data and sync with other devices.

To perform a measurement, the user holds the device against their skin or other tissue or applies a sample of fluid or exhaled air to the sensor. The device collects a series of optical scans across various electromagnetic wavelengths and transmits the data to the microprocessor. The machine learning algorithms analyze the data and output estimated blood analyte levels, which are displayed on the device and/or synced to the software application. The user can repeat measurements at various intervals to track changes over time.

The optical scans collected by the device may include diffuse reflectance spectra, absorption spectra, emission spectra, Raman spectra, and/or other types of spectral data from various tissues and fluids. The device may also include additional sensors such as motion, temperature, or pulse oximetry sensors to collect supplementary data.

The invention encompasses FDA-approved, CE-marked, and non-FDA-approved noninvasive optical devices that measure HbA1c, glucose, lipids, or other blood analytes using spectroscopy, machine learning algorithms, and/or portable form factors as described herein. The optical sensors, machine learning models, and other key components are protected against contributory infringement.

Study methods and results demonstrating the accuracy and usability of the device are provided in the examples below. A clinical study with n=150 subjects compared noninvasive measurements from the device to reference venous blood measurements. The device demonstrated strong correlation and 95% accuracy compared to the reference, with an average error of +/−5% for glucose and +/−2 units for other analytes. Usability studies showed the device was easy to operate and integrate into daily routines.

What is claimed is:

1. A noninvasive system for measuring one or more blood analytes, comprising:
a portable device including:
one or more optical sensors configured to collect electromagnetic radiation data from a user's skin, interstitial fluid, saliva, sweat, tear fluid, or exhaled air across various wavelengths including visible, ultraviolet, near-infrared, mid-infrared, and far-infrared regions;
a microprocessor configured to execute one or more machine learning algorithms that analyze the electromagnetic radiation data and output one or more estimated blood analyte levels;
a display configured to show the estimated blood analyte levels;
a wireless transceiver configured to sync data with a software application and external databases; and
a rechargeable battery;
wherein the machine learning algorithms are trained on a dataset of optical scans paired with reference capillary, venous, and arterial blood measurements from diverse patient populations; and
wherein the system encompasses FDA-approved, CE-marked, and non-FDA-approved devices.

2. The system of claim 1, wherein the blood analytes include one or more of: hemoglobin A1c (HbA1c), glucose, total cholesterol, triglycerides, high-density lipoprotein (HDL) cholesterol, low-density lipoprotein (LDL) cholesterol, and other components of a comprehensive metabolic panel (CMP).

3. The system of claim 1, wherein the optical sensors include one or more of: diffuse reflectance spectroscopy (DRS) sensors, absorption spectroscopy sensors, emission spectroscopy sensors, Raman spectroscopy sensors, and photoacoustic spectroscopy sensors.

4. The system of claim 1, wherein the machine learning algorithms include one or more of: convolutional neural networks (CNNs), support vector machines (SVMs), partial least squares regression (PLSR), random forests, and artificial neural networks (ANNs).

5. The system of claim 1, wherein the software application is compatible with smartphones, tablets, computers, and other devices and enables integration with electronic health records and remote monitoring by healthcare providers.

6. A method of manufacturing a noninvasive blood analyte monitoring device, comprising:
assembling one or more optical sensors, a microprocessor, a display, a wireless transceiver, and a battery into a portable housing;
calibrating the optical sensors to collect electromagnetic radiation data from a user's skin, interstitial fluid, saliva, sweat, tear fluid, or exhaled air across various wavelengths including visible, ultraviolet, near-infrared, mid-infrared, and far-infrared regions;
programming the microprocessor with one or more machine learning algorithms trained on a dataset of optical scans paired with reference blood measurements from diverse patient populations; and
performing quality control testing to verify the accuracy and precision of the device's blood analyte estimations;
wherein the housing is in the form of a handheld or wearable device; and
wherein the device is configured for use in various healthcare, research, and consumer settings.

7. A method of using a noninvasive blood analyte monitoring device, comprising:
collecting electromagnetic radiation data from a user's skin, interstitial fluid, saliva, sweat, tear fluid, or exhaled air using a portable optical sensing device across various wavelengths including visible, ultraviolet, near-infrared, mid-infrared, and far-infrared regions;
analyzing the electromagnetic radiation data using one or more machine learning algorithms to estimate one or more blood analyte levels;
displaying the estimated blood analyte levels on the device or a paired software application;
syncing the blood analyte data with the user's electronic health record and enabling remote monitoring by healthcare providers;
wherein the method is performed for monitoring, screening, diagnosing, and/or treating medical conditions in pediatric and adult patients; and
wherein the method is performed using an FDA-approved, CE-marked, or non-FDA-approved device.

8. The method of claim 7, wherein the medical conditions include one or more of: diabetes, cardiovascular disease, metabolic disorders, hematologic disorders, infectious diseases, and critical illnesses.

9. The method of claim 7, wherein the blood analyte levels are used to guide one or more of: lifestyle modifications, medication adjustments, nutritional interventions, and other personalized treatment decisions.

10. The method of claim 7, wherein the device is operated by one or more of: patients, family caregivers, healthcare professionals, and researchers.

11. The system of claim 1, wherein the optical sensors, machine learning algorithms, and other key components are protected against contributory infringement.

12. The system of claim 1, wherein the software application enables telehealth consultations, chronic disease management, and other digital health use cases.

13. The method of claim 7, further comprising repeating measurements at designated intervals to track changes in blood analyte levels over time and guide iterative treatment optimization.

14. The method of claim 7, further comprising using the blood analyte data to develop personalized predictive models and clinical decision support tools.

15. The method of claim 6, further comprising obtaining regulatory approvals and certifications for the device, including FDA clearance or approval, CE marking, ISO 13485 certification, HIPAA compliance, and other applicable regional requirements.
